(19) United States
(12) Patent Application Publication
(10) Pub. No.: US 2024/0018478 A1
(43) Pub. Date: Jan. 18, 2024

(54) SELF-ORGANISED HUMAN CARDIAC ORGANOID

(71) Applicant: GENOME BIOLOGICS UG, Kronberg im Taunus (DE)

(72) Inventors: Duc Minh PHAM, Offenbach (DE); Jaya KRISHNAN, Kronberg im Taunus (DE); Stefanie DIMMELER, Frankfurt am Main (DE)

(73) Assignee: GENOME BIOLOGICS UG, Kronberg im Taunus (DE)

(21) Appl. No.: 18/256,230

(22) PCT Filed: Dec. 7, 2021
(86) PCT No.: PCT/EP2021/084656

§ 371 Date: Jun. 7, 2023

(30) Foreign Application Priority Data
Dec. 7, 2020 (EP) 20212131.5
Dec. 17, 2020 (EP) 20214885.4
Jun. 8, 2021 (EP) 21178359.2

(51) Int. Cl. C12N 5/077

(52) U.S. Cl. CPC C12N 5/0657; C12N 2501/115; C12N 2506/45; C12N 2501/15; C12N 2501/33; C12N 2503/02; C12N 2501/40; C12N 2501/155; C12N 2513/00; C12N 2501/16

(57) ABSTRACT

The present invention relates to a preparation method for a self-organized cardiac organoid. The invention also relates to a cardiac tissue organoid obtained by the method and to its use in regenerative medicine, as a tissue implant, or in drug screening.

The present invention relates to a preparation method for a self-organized cardiac organoid. The invention also relates to a cardiac tissue organoid obtained by the method and to its use in regenerative medicine, as a tissue implant, or in drug screening.

### BACKGROUND OF THE INVENTION

Cardiac tissue engineering promises to create therapeutic tissue replacements for repair of the diseased native myocardium and as an ethical non-animal alternative for drug testing.

Based on the above-mentioned state of the art, the objective of the present invention is to provide means and methods to producing a cardiac tissue organoid. This objective is attained by the subject-matter of the independent claims of the present specification, with further advantageous embodiments described in the dependent claims, examples, figures and general description of this specification.

### SUMMARY OF THE INVENTION

The inventors herein provide a method for the generation of functional cardiac tissue organoids comprising human endothelial cells (EC), cardiac fibroblasts (FB) and cardiomyocytes (CM) differentiated directly from human induced pluripotent stem (hiPS) cells. These organoids contain a 3D blood vascular network with pericyte and/or smooth muscle cell coverage, spontaneously organise into epicardial, myocardial and endocardial layers, exhibit a distinct lumen, and mimic human myocardial responses to stress stimulation at the molecular, biochemical and physiologic level. Thus, these organoids could serve as an immediate alternative to monoculture 2D system as a more precise context for drug testing and validation. Such organoids may serve as an autologous tissue source for the replacement and repair of damaged heart tissue.

A first aspect of the invention relates to a method for production of a cardiac tissue organoid. The method comprises the steps:
a) providing pluripotent stem cells in a well or a microwell, wherein the well or microwell comprises medium 0, and incubating.
b) subsequently, replacing the medium with medium 1, and incubating.
c) subsequently, replacing the medium with medium 2, and incubating.
d) subsequently, replacing the medium with medium 3, and incubating.
e) subsequently, repeating step d of incubating with medium 3 one time.
f) subsequently, replacing the medium with medium 4, and incubating.
g) repeating step f of incubating with medium 4 for 4-6 times.
h) subsequently, replacing the medium with medium 5, and incubating.
i) subsequently, transferring cells to a rotating incubator and replacing the medium with medium 6, and incubating and replacing the medium regularly.
j) keeping the cells in the rotating incubator.
k) harvesting the cardiac tissue organoid.

A second aspect of the invention relates to a cardiac tissue organoid obtained by the method of the first aspect.

A third aspect of the invention relates to the cardiac tissue organoid according to the second aspect use as a tissue implant or for use in regenerative medicine.

A fourth aspect of the invention relates to a method for screening a drug comprising the steps:
producing a cardiac tissue organoid according to the method of the first aspect,
contacting the cardiac tissue organoid with a drug of interest,
determining an effect of the drug on the cardiac tissue organoid.

### DETAILED DESCRIPTION OF THE INVENTION

Terms and Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 4th ed. (2012) Cold Spring Harbor Laboratory Press, Cold Spring Harbor, N.Y. and Ausubel et al., Short Protocols in Molecular Biology (2002) 5th Ed, John Wiley & Sons, Inc.) and chemical methods.

The term ACS-2-P in the context of the present specification relates to L-ascorbic acid 2 phosphate sesquimagnesium salt hydrate (CAS No: 113170-55-1). ACS-2-P enhances card iomyocyte differentiation.

The term Activin A in the context of the present specification relates to recombinant Activin A Protein (UniProt-ID: P08476). Activin A is essential for mesoderm induction.

The term BMP-4 in the context of the present specification relates to recombinant BMP-4 Protein (UniProt-ID: P12644). BMP-4 is essential for mesoderm induction.

The term CHIR in the context of the present specification relates to 6-[[2-[[4-(2,4-Dichlorophenyl)-5-(5-methyl-1H-imidazol-2-yl)-2-pyrimidinyl]amino]ethyl]amino]-3-pyridinecarbonitrile (CAS-No: 252917-06-9). CHIR is a glycogen synthase kinase (GSK) 3 inhibitor/Wnt activator, and enhances mesoderm induction.

The term FGF in the context of the present specification relates to recombinant fibroblast growth factor 2 (UniProt-ID: P09038). FGF enhances mesoderm induction and also enhances endothelial cells differentiation, which is important for vascularization.

The term hFGF in the context of the present specification relates to recombinant human fibroblast growth factor 2 (UniProt-ID: P09038).

The term VEGF in the context of the present specification relates to recombinant VEGF (Vascular Endothelial Growth Factor) 165 Protein (UniProt-ID: P15692). VEGF enhances endothelial cells differentiation, which is important for vascularization.

The term hVEGF in the context of the present specification relates to recombinant human VEGF 165 Protein (UniProt-ID: P15692).

The term IWP-4 in the context of the present specification relates to N-(6-Methyl-2-benzothiazolyl)-2-[(3,4,6,7-tetrahydro-3-(2-methoxyphenyl)-4-oxothieno[3,2-d]pyrimidin-2-yl)thio]-acetamide (CAS-No: 686772-17-8). IWP-4 is a potent inhibitor of the Wnt/p-catenin signalling, which makes it essential for cardiac differentiation.

The term TGFβ1 in the context of the present specification relates to transforming growth factor beta 1 (UniProt-ID: P01137). TGFβ1 is required to maintain the pluripotency of the stem cells.

Insulin has the UniProt-ID: P01308. Insulin improves the seeding and proliferating of stem cells.

The term EGF in the context of the present specification relates to epidermal growth factor (UniProt-ID: P01133). EGF accelerates angiogenesis.

The term IGF in the context of the present specification relates to insulin-like growth factor 1 (UniProt-ID: Q13429). IGF promotes migration and tube formation of endothelial cells.

The term FBS in the context of the present specification relates to fetal bovine serum.

Ascorbic acid (CAS-No: 50-81-7) enhances endothelial synthesis and supports vascular formation.

Heparin (CAS-No: 9005-49-6) stimulates the proliferation of endothelial cells.

Hydrocortisone (CAS-No: 50-23-7) sensitizes endothelial cells to growth factors and increases proliferation.

A first aspect of the invention relates to a method for production of a cardiac tissue organoid.

The method comprises the steps:
a) providing pluripotent stem cells in a well or a microwell, wherein the well or microwell comprises medium 0 (Medium TeSR™-E8™), and incubating. In certain embodiments, the incubation is for 44-52 h. In certain embodiments, the incubation is for 46-50 h. In certain embodiments, the incubation is for ˜48 h.
b) subsequently, replacing the medium with medium 1, and incubating. In certain embodiments, the incubation is for 44-52 h. In certain embodiments, the incubation is for 46-50 h. In certain embodiments, the incubation is for ˜48 h.
c) subsequently, replacing the medium with medium 2, and incubating. In certain embodiments, the incubation is for 44-52 h. In certain embodiments, the incubation is for 46-50 h. In certain embodiments, the incubation is for ˜48 h.
d) subsequently, replacing the medium with medium 3, and incubating. In certain embodiments, the incubation is for 44-52 h. In certain embodiments, the incubation is for 46-50 h. In certain embodiments, the incubation is for ˜48 h.
e) subsequently, repeating step d of incubating with medium 3 one time. In certain embodiments, the incubation is for 44-52 h. In certain embodiments, the incubation is for 46-50 h. In certain embodiments, the incubation is for ˜48 h.
f) subsequently, replacing the medium with medium 4, and incubating. In certain embodiments, the incubation is for 44-52 h. In certain embodiments, the incubation is for 46-50 h. In certain embodiments, the incubation is for ˜48 h.
g) repeating step f of incubating with medium 4 for 4-6 times. In certain embodiments, the incubation is repeated 5 times. In certain embodiments, each incubation is for 44-52 h. In certain embodiments, each incubation is for 46-50 h. In certain embodiments, each incubation is for ˜48 h.
h) subsequently, replacing the medium with medium 5, and incubating. In certain embodiments, each incubation is for 44-52 h. In certain embodiments, each incubation is for 46-50 h. In certain embodiments, each incubation is for ˜48 h.
i) subsequently, transferring cells to a rotating incubator and replacing the medium with medium 6, and incubating. In certain embodiments, the incubation is repeated 5 times. In certain embodiments, each incubation is for 44-52 h. In certain embodiments, each incubation is for 46-50 h. In certain embodiments, each incubation is for ˜48 h.
j) keeping the cells in the rotating incubator. In certain embodiments, the cells are kept in the incubator for a period of 5 to 7 days. In certain embodiments, the cells are kept in the incubator for a period of ˜6 days. For the cells in the incubator, there is a continuous flow of fresh medium 6 or the medium is replaced regularly with fresh medium 6. In certain embodiments, medium 6 is replaced 2-4 times. In certain embodiments, medium 6 is replaced 3 times. In certain embodiments, each medium replacement after 44-52 h. In certain embodiments, each medium replacement after 46-50 h. In certain embodiments, each medium replacement after ˜48 h.
k) harvesting the cardiac tissue organoid.

All the incubation steps are performed in an incubator with 37° C. and 5% CO₂ Also, the rotating incubator is at 37° C. and 5% CO₂ When the medium is replaced, it is understood that the only the medium is removed and new medium is added, while the cells remain in the well or microwell.

In certain embodiments, the pluripotent stem cells are induced pluripotent stem cells (iPSCs).

Medium 0 is Medium TeSa™-E8™ (https://www.stemcell.com/products/tesr-e8.html). Medium 0 comprises: FGF2, insulin, and TGFβ1, and medium 0 does not comprise: Activin A, BMP-4, CHIR, ACS-2-P, IWP4, and VEGF.

In certain embodiments, medium 0 comprises
30-250 μg/L FGF2,
10-30 mg/L insulin, and
0.5-5 μg/L TGF81.

In certain embodiments, medium 0 comprises
50-200 μg/L FGF2,
15-25 mg/L insulin, and
1-3 μg/L TGF81.

In certain embodiments, medium 0 comprises
˜100 μg/L FGF2,
˜20 mg/L insulin, and
˜2 μg/L TGF81.

In certain embodiments, medium 0 comprises 30-250 μg/L FGF2. In certain embodiments, medium 0 comprises 10-30 mg/L insulin. In certain embodiments, medium 0 comprises 0.5-5 μg/L TGF81. In certain embodiments, medium 0 comprises 50-200 μg/L FGF2. In certain embodiments, medium 0 comprises 15-25 mg/L insulin. In certain embodiments, medium 0 comprises 1-3 μg/L TGF81. In certain embodiments, medium 0 comprises ˜100 μg/L FGF2. In certain embodiments, medium 0 comprises ˜20 mg/L insulin. In certain embodiments, medium 0 comprises ˜2 μg/L TGF81.

Medium 1 comprises: Activin A, BMP-4, FGF (particularly hFGF), CHIR, and ACS-2-P; and medium 1 does not comprise: IWP4, VEGF, insulin, and TGF81. Medium 1 is supplemented with a solubilized membrane preparation extracted from mammalian cells (Matrigel).

Matrigel is Corning® Matrigel® hESC-Qualified Matrix, LDEV-free, #354277 (Corning) (available from https://ecatalog.corninq.com/life-sciences/b2c/US/en/Surfaces/Extracellular-Matrices-ECMs/Corning % C2%AE-Matrigel®/0C2%AE-Matrix/p/354277). Corning Matrigel matrix is a solubilized basement membrane preparation extracted from the Engelbreth-Holm-Swarm (EHS) mouse sarcoma, a tumor rich in extracellular matrix proteins, including Laminin (a major component), Collagen IV, heparan sulfate proteoglycans, entactin/nidogen, and a number of growth factors.

In certain embodiments, medium 1 comprises
30-70 ng/ml Activin A,
0.5-4 ng/ml BMP-4,
3-7 ng/ml FGF,
0.25-3 μmol/L CHIR, and
25-200 μmol/L ASC-2-P.

In certain embodiments, medium 1 comprises
40-60 ng/ml Activin A,
1-3 ng/ml BMP-4,
4-6 ng/ml FGF,
0.5-2 μmol/L CHIR, and
50-150 μmol/L ASC-2-P.

In certain embodiments, medium 1 comprises
˜50 ng/ml Activin A,
˜2 ng/ml BMP-4,
˜5 ng/ml FGF,
˜1 μmol/L CHIR, and
˜100 μmol/L ASC-2-P.

In certain embodiments, medium 1 comprises 30-70 ng/ml Activin A. In certain embodiments, medium 1 comprises 0.5-4 ng/ml BMP-4. In certain embodiments, medium 1 comprises 3-7 ng/ml FGF. In certain embodiments, medium 1 comprises 0.25-3 μmol/L CHIR. In certain embodiments, medium 1 comprises 25-200 μmol/L ASC-2-P. In certain embodiments, medium 1 comprises 40-60 ng/ml Activin A. In certain embodiments, medium 1 comprises 1-3 ng/ml BMP-4. In certain embodiments, medium 1 comprises 4-6 ng/ml FGF. In certain embodiments, medium 1 comprises 0.5-2 μmol/L CHIR. In certain embodiments, medium 1 comprises 50-150 μmol/L ASC-2-P. In certain embodiments, medium 1 comprises ˜50 ng/ml

Activin A. In certain embodiments, medium 1 comprises ˜2 ng/ml BMP-4. In certain embodiments, medium 1 comprises ˜5 ng/ml FGF. In certain embodiments, medium 1 comprises ˜1 μmol/L CHIR. In certain embodiments, medium 1 comprises ˜100 μmol/L ASC-2-P.

Medium 2 comprises: Activin A at a concentration which is lower than in medium 1, BMP-4 at a concentration which is higher than in medium 1, FGF, particularly hFGF, CHIR, and ACS-2-P, and medium 2 does not comprise: IWP4, VEGF, insulin, and TGFβ1.

In certain embodiments, Activin A in medium 2 is at a concentration which is 5-20% (mass/volume) of the concentration of Activin A in medium 1. In certain embodiments, Activin

A in medium 2 is at a concentration which is 8-15% (m/v) of the concentration of Activin A in medium 1. In certain embodiments, Activin A in medium 2 is at a concentration which is ˜10% (m/v) of the concentration of Activin A in medium 1.

In certain embodiments, BMP-4 in medium 2 is at a concentration which is 500-2000% (m/v) of the concentration of BMP-4 in medium 1. In certain embodiments, BMP-4 in medium 2 is at a concentration which is 800-1300% (m/v) of the concentration of BMP-4 in medium 1. In certain embodiments, BMP-4 in medium 2 is at a concentration which is ˜1000% (m/v) of the concentration of BMP-4 in medium 1.

In certain embodiments, medium 2 comprises
3-7 ng/ml Activin A,
4-20 ng/ml BMP-4,
3-7 ng/ml FGF,
0.25-3 μmol/L CHIR, and
25-200 μmol/L ASC-2-P.

In certain embodiments, medium 2 comprises
4-6 ng/ml Activin A,
5-15 ng/ml BMP-4,
4-6 ng/ml FGF,
0.5-2 μmol/L CHIR, and
50-150 μmol/L ASC-2-P.

In certain embodiments, medium 2 comprises
˜5 ng/ml Activin A,
˜10 ng/ml BMP-4,
˜5 ng/ml FGF,
˜1 μmol/L CHIR, and
˜100 μmol/L ASC-2-P.

In certain embodiments, medium 2 comprises 3-7 ng/ml Activin A. In certain embodiments, medium 2 comprises 4-20 ng/ml BMP-4. In certain embodiments, medium 2 comprises 3-7 ng/ml FGF. In certain embodiments, medium 2 comprises 0.25-3 μmol/L CHIR. In certain embodiments, medium 2 comprises 25-200 μmol/L ASC-2-P. In certain embodiments, medium 2 comprises 4-6 ng/ml Activin A. In certain embodiments, medium 2 comprises 5-15 ng/ml BMP-4. In certain embodiments, medium 2 comprises 4-6 ng/ml FGF. In certain embodiments, medium 2 comprises 0.5-2 μmol/L CHIR. In certain embodiments, medium 2 comprises 50-150 μmol/L ASC-2-P. In certain embodiments, medium 2 comprises ˜5 ng/ml Activin A. In certain embodiments, medium 2 comprises ˜10 ng/ml BMP-4. In certain embodiments, medium 2 comprises ˜5 ng/ml FGF. In certain embodiments, medium 2 comprises ˜1 μmol/L CHIR. In certain embodiments, medium 2 comprises ˜100 μmol/L ASC-2-P.

Medium 3 comprises: IWP4, insulin, and ACS-2-P, and medium 3 does not comprise: VEGF, Activin A, BMP-4, FGF, CHIR, and TGF81.

In certain embodiments, medium 3 comprises
2-8 μmol/L IWP4, and
50-400 μmol/L ACS-2-P.

In certain embodiments, medium 3 comprises
3.5-6.5 μmol/L IWP4, and
100-300 μmol/L ACS-2-P.

In certain embodiments, medium 3 comprises
˜5 μmol/L IWP4, and
˜200 μmol/L ACS-2-P.

In certain embodiments, medium 3 comprises 2-8 μmol/L IWP4. In certain embodiments, medium 3 comprises 50-400 μmol/L ACS-2-P. In certain embodiments, medium 3 comprises 3.5-6.5 μmol/L IWP4. In certain embodiments, medium 3 comprises 100-300 μmol/L ACS-2-P. In certain embodiments, medium 3 comprises ˜5 μmol/L IWP4. In certain embodiments, medium 3 comprises ˜200 μmol/L ACS-2-P.

Medium 4 comprises: ACS-2-P, and insulin, and medium 4 does not comprise: IWP4, VEGF, Activin A, BMP-4, FGF, CHIR, and TGF81.

In certain embodiments, medium 4 comprises
50-400 μmol/L ACS-2-P.

In certain embodiments, medium 4 comprises
100-300 μmol/L ACS-2-P.

In certain embodiments, medium 4 comprises
˜200 μmol/L ACS-2-P.

Medium 5 comprises: ACS-2-P, insulin, VEGF (particularly hVEGF), and FGF (particularly hFGF), and medium 5 does not comprise: IWP4, Activin A, BMP-4, CHIR, and TGF61.

In certain embodiments, medium 5 comprises
50-400 μmol/L ACS-2-P,
20-100 nmol/L VEGF, and
5-100 nmol/L FGF.

In certain embodiments, medium 5 comprises
100-300 μmol/L ACS-2-P,
30-70 nmol/L VEGF, and
10-50 nmol/L FGF.

In certain embodiments, medium 5 comprises
˜200 μmol/L ACS-2-P,
˜50 nmol/L VEGF, and
˜25 nmol/L FGF.

In certain embodiments, medium 5 comprises 50-400 μmol/L ACS-2-P. In certain embodiments, medium 5 comprises 20-100 nmol/L VEGF. In certain embodiments, medium 5 comprises 5-100 nmol/L FGF. In certain embodiments, medium 5 comprises 100-300 μmol/L ACS-2-P. In certain embodiments, medium 5 comprises 30-70 nmol/L VEGF. In certain embodiments, medium 5 comprises 10-50 nmol/L FGF. In certain embodiments, medium 5 comprises ˜200 μmol/L ACS-2-P. In certain embodiments, medium 5 comprises ˜50 nmol/L VEGF. In certain embodiments, medium 5 comprises ˜25 nmol/L FGF.

Medium 6 comprises: ACS-2-P, insulin, and Endothelial Cell Growth Medium 2 comprising EGF, FGF, IGF, and VEGF, and medium 6 does not comprise: IWP4, Activin A, BMP-4, CHIR, and TGF61. In certain embodiments, medium 6 also comprises: ascorbic acid, heparin; and hydrocortisone.

Endothelial Cell Basal Medium and Endothelial Cell Growth Medium Supplement Pack are purchasable from PromoCell (https://.promocell.com/oroduct/endothelial-cell-crowth-medium-2/). It is a cell culture medium for endothelial cells from large blood vessels.

In certain embodiments, medium 6 comprises
30-350 μmol/L ACS-2-P,
1-15 ng/ml EGF;
3-20 ng/ml FGF;
10-30 ng/ml IGF; and
0.1-1.5 ng/ml VEGF.

In certain embodiments, medium 6 comprises
80-250 mol/L ACS-2-P,
3-10 ng/ml EGF;
5-15 ng/ml FGF;
15-25 ng/ml IGF; and
0.3-1 ng/ml VEGF.

In certain embodiments, medium 6 comprises
˜160 μmol/L ACS-2-P,
˜5 ng/ml EGF;
˜10 ng/ml FGF;
˜20 ng/ml IGF; and
˜0.5 ng/ml VEGF.

In certain embodiments, medium 6 comprises 30-350 μmol/L ACS-2-P. In certain embodiments, medium 6 comprises 1-15 ng/ml EGF. In certain embodiments, medium 6 comprises 3-20 ng/ml FGF. In certain embodiments, medium 6 comprises 10-30 ng/ml IGF. In certain embodiments, medium 6 comprises 0.1-1.5 ng/ml VEGF. In certain embodiments, medium 6 comprises 80-250 mol/L ACS-2-P. In certain embodiments, medium 6 comprises 3-10 ng/ml EGF. In certain embodiments, medium 6 comprises 5-15 ng/ml FGF. In certain embodiments, medium 6 comprises 15-25 ng/ml IGF. In certain embodiments, medium 6 comprises 0.3-1 ng/ml VEGF. In certain embodiments, medium 6 comprises ˜160 μmol/L ACS-2-P. In certain embodiments, medium 6 comprises ˜5 ng/ml EGF. In certain embodiments, medium 6 comprises ˜10 ng/ml FGF. In certain embodiments, medium 6 comprises ˜20 ng/ml IGF. In certain embodiments, medium 6 comprises ˜0.5 ng/ml VEGF.

In certain embodiments, medium 6 additionally comprises ˜1 μg/ml ascorbic acid, ˜22.5 μg/ml heparin, and ˜0.2 μg/ml hydrocortisone.

In certain embodiments, each medium 1 to 6 comprises
RPMI 1640 supplemented with a vitamin and amino acid mix (Glutamax),
˜1 mMol/L sodium pyruvate,
˜100 U/mL penicillin,
˜100 μg/mL streptomycin, and
˜3% (volume/volume) of a cell growth supplement (B27 supplement).

RPMI 1640 is purchasable from ThermoFisher

(https://.thermofisher.com/de/de/home/life-science/cell-culture/mammalian-cell-culture/classical-media/rpmi.html). RPMI 1640 Medium is unique from other media because it contains the reducing agent glutathione and high concentrations of vitamins. RPMI 1640 Medium contains biotin, vitamin B12, and para-aminobenzoic acid.

Glutamax is purchasable from ThermoFisher

(https://www.thermofisher.comide/de/home/technical-resources/media-formulation.122.html). It comprises amino acids, vitamins, inorganic salts, and glucose.

B27 supplement is purchasable from ThermoFisher

(https://www.thermofishercom/de/de/home/brands/gibco/qibco-b-27-supplement.html). It comprises Catalase, Glutathione reduced, Human Insulin, Superoxide Dismutase, Human Holo-Transferin, T3 (Triiodo-L-Thyronine), L-carnitine, Ethanolamine, D+-galactose, Putrescine, Sodium selenite, Corticosterone, Linoleic acid, Linolenic acid, Progesterone, Retinol acetate, DL-alpha tocopherol, DL-alpha tocopherol acetate, Oleic acid, Pipecolic acid, and Biotin.

In certain embodiments, in step a of providing the iPSCs, 100.000-600.000 cells/well or 500-2000 cells/microwell are provided.

A second aspect of the invention relates to a cardiac tissue organoid obtained by the method of the first aspect.

An alternative of the second aspect of the invention relates to a cardiac tissue organoid obtainable by the method of the first aspect.

In certain embodiments, the cardiac tissue organoid comprises cardiomyocytes;
endothelial cells;
fibroblasts;
smooth muscle cells;
pericytes;
neurons/sino-atrial node cells; and
macrophages.

In certain embodiments, the cardiac tissue organoid comprises the following proportions by cell number
46.6% (±10.2%) cardiomyocytes;
8.0% (±1.3%) endothelial cells;
45.4% (±10.2%) fibroblasts;
>3% smooth muscle cells;
>3% pericytes;
>1% neurons/sino-atrial node cells; and
>1% macrophages.

These numbers are in line with previously published data for the human heart cell composition by cell number: Litvinukova, M. et al. Cells of the adult human heart. Nature (2020) doi:10.1038/s41586-020-2797-4.

In certain embodiments, the cardiac tissue organoid comprises the following proportions by cell area
˜68.7% (±7.1%) cardiomyocytes;
˜6.3% (±1.5%) endothelial cells;
˜25.3% (±8.1%) fibroblasts;
>2% smooth muscle cells;
>2% pericytes;
>1% neurons/sino-atrial node cells; and
>1% macrophages.

These numbers are in line with previously published data for the human heart cell composition: Zhou, P. & Pu, W. T. Recounting Cardiac Cellular Composition. Circ. Res. 118, 368-370 (2016).

Both cell surface and internal markers are used to identify respective cell lineages:
cardiomyocytes (heart cells): sarcomeric α-actinin, myosin heavy chain 6 and 7, myosin-binding protein C, and titin
endothelial cells: VE-cadherin, PECAM, Isolectin IB4
fibroblasts: phalloidin (stains Actin)
smooth muscle cells/pericytes: smooth muscle actin, Neural/glial antigen 2 (NG2)
neurons/sino-atrial node cells: Hyperpolarization-activated cyclic nucleotide-gated potassium channel 4 (HCN4), Pan-neuronal markers (comprising antigens of somatic, nuclear, dendritic, and axonal proteins)
macrophages: CD11 b, CD68.

In certain embodiments, the cardiac tissue organoid is in a mature phenotypic status characterized by
a rod shape;
a polarized structure; and
a sarcomeric structure of cardiomyocytes.

In certain embodiments, the cardiac tissue organoid is in a mature structural status characterized by a decreased ratio of mRNA expression levels of the mature cardiac organoid (day 35 after cardiomyocytes were made) relative to an immature cardiac organoid (d0—the first day after cardiomyocytes were made) for the following markers:
MYH6/MYH7, particularly at a ratio of 1:21.1;
MLC2a/MLC2v, particularly at a ratio of 1:21.001;
TNNI1/TNNI3, particularly at a ratio of 1:21.3;
and
TTN-N2B/TTN-N2BA, particularly at a ratio of 121.01.

Switching isoforms of genes is related to myofibril assembly towards an adult-type pattern.

In certain embodiments, the cardiac tissue organoid is in a mature functional status characterized by mRNA expression level of CAV3 (normalized to HPRT) of 0.01. In certain embodiments, the cardiac tissue organoid is in a mature functional status characterized by mRNA expression level of CAV3 (normalized to HPRT) of 0.04.

In certain embodiments, the cardiac tissue organoid is in a mature metabolic status characterized by an mRNA expression level of PKM2 (normalized to HPRT) of 2. In certain embodiments, the cardiac tissue organoid is in a mature metabolic status characterized by an mRNA expression level of 0.8.

In certain embodiments, the cardiac tissue organoid comprises
epicardium;
myocardium;
endocardium;
cardiac lumen; and
a three-dimensional vascular network.

These organoids contain a 3D blood vascular network with pericyte and/or smooth muscle cell coverage, spontaneously organise into epicardial, myocardial and endocardial layers, exhibit a distinct lumen, and mimic human myocardial responses to stress stimulation at the molecular, biochemical and physiologic level.

The tissue types are determined as defined and detailed in: Saxton, A., Tariq, M. A. & Bordoni, B. Anatomy, Thorax, Cardiac Muscle. StatPearls (2020), and LeGrice et al. (1995) Heart and Circulatory Physiology, Volume 269, Issue 2.

Demarcation of the respective cardiac layers was performed by combinatorial antibody staining using lineage-specific markers (as listed on page 13) and confocal microscopic imaging as previously described (Velecela et al. Development. 2019 Oct. 17; 146(20):dev178723.; Seidel et al. Ann Biomed Eng. 2016 May; 44(5):1436-1448.; Marron et al. Cardiovasc Res. 1994 October;28(10):1490-9.). Epicardial, myocardial and endocardial patterning was consistently observed across generated organoids.

In certain embodiments, the cardiac tissue organoid consists of human cells.

A third aspect of the invention relates to the cardiac tissue organoid according to the second aspect use as a tissue implant or for use in regenerative medicine.

A fourth aspect of the invention relates to a method for screening a drug comprising the steps:
producing a cardiac tissue organoid according to the method of the first aspect,
contacting the cardiac tissue organoid with a drug of interest,
determining an effect of the drug on the cardiac tissue organoid.

In certain embodiments, the cardiac tissue organoid may be subjected to chemical stress (e.g. toxic compounds), mechanical stress, nutrient stress (e.g. over nutrition, malnutrition, starvation), environmental stress (e.g. hyperoxia, hypoxia, heat) or genetic stress (e.g. siRNA or shRNA mediated gene knowdown, CRISPR/Cas gene editing, mutagenesis and deletion, recombination based gene editing, mutagenesis and deletion) to mimic disease drivers.

In certain embodiments, the effect of the drug on the cardiac tissue organoid is determined as one or several effects selected from:
Cell viability;
Cytotoxicity;
Cardiomyocyte cell death;
Proliferation;
Contractility;
Mitochondrial activity;
Metabolism.

Parameters are assayed as follows:
Cell viability: AlamarBlue
Cytotoxicity: LDH assay, γ-H2AX staining (for DNA damage)
Cardiomyocyte cell death: (high-sensitive cardiac Troponin T assay (hsTNT) or cardiac Troponin T assay (cTNT), cleaved-Caspase 3 staining
Proliferation: EdU, and Ki-67, phospho-Histone H3, Aurora B staining
Contractility: Organoid beating rate, lonoptix—for contractile frequency and amplitude, and calcium (Ca2+) flux
Mitochondrial activity: JC10, Mitotracker, TMRE-Mitochondrial Membrane Potential Assay Kit
Metabolism: SeaHorse assay (looking at Organoid Glycolysis, Oxidative phosphorylation, Fatty acid oxidation and Pentose Phosphate Pathway rates), lactate secretion (as measure for Glycolysis).

Items:

The invention further encompasses the following embodiments, designated “Items” in the following:
1. A method for production of a cardiac tissue organoid comprising the steps:
a) providing pluripotent stem cells, particularly induced pluripotent stem cells (iPSCs), in a well or a microwell, wherein the well or microwell comprises medium 0, and incubating said cells for 44-52 h, particularly for 46-50 h, more particularly for ˜48 h, wherein
   medium 0 comprises:
      FGF2,
      insulin, and
      TGFβ1
b) subsequently, replacing the medium with medium 1, wherein
   medium 1 comprises
      Activin A,
      BMP-4,
      FGF, particularly hFGF,
      CHIR, and
      ACS-2-P;
   medium 1 is supplemented with a solubilized membrane preparation extracted from mammalian cells (Matrigel), and incubating for 44-52 h, particularly for 46-50 h, more particularly for ˜48 h,
c) subsequently, replacing the medium with medium 2, wherein
   medium 2 comprises
      Activin A at a concentration which is lower than in medium 1, particularly Activin A at a concentration which is 5-20% (mass/volume), particularly 8-15% (m/v), more particularly ˜10% (m/v) of the concentration of Activin A in medium 1; BMP-4 at a concentration which is higher than in medium 1, particularly BMP-4 at a concentration which is 500-2000% (m/v), particularly 800-1300% (m/v), more particularly ˜1000% (m/v) of the concentration of BMP-4 in medium 1;
      FGF, particularly hFGF,
      CHIR, and
      ACS-2-P;
and incubating for 44-52 h, particularly for 46-50 h, more particularly for ˜48 h,
d) subsequently, replacing the medium with medium 3, wherein
   medium 3 comprises
      insulin,
      IWP4, and
      ACS-2-P;
and incubating for 44-52 h, particularly for 46-50 h, more particularly for ˜48 h,
e) subsequently, repeating step d of incubating with medium 3 one time,
f) subsequently, replacing the medium with medium 4, wherein
   medium 4 comprises
      insulin, and
      ACS-2-P;
and incubating for 44-52 h, particularly for 46-50 h, more particularly for ˜48 h,
g) repeating step f of incubating with medium 4 for 4-6 times, particularly 5 times;
h) subsequently, replacing the medium with medium 5, wherein
   medium 5 comprises
      insulin,
      ACS-2-P,
      VEGF, particularly hVEGF, and
      FGF, particularly hFGF;
and incubating for 44-52 h, particularly for 46-50 h, more particularly for ˜48 h,
i) subsequently, transferring cells to a rotating incubator and replacing the medium with medium 6, wherein
   medium 6 comprises
      insulin,
      ACS-2-P, and
      Endothelial Cell Growth Medium 2 comprising
         EGF;
         FGF;
         IGF; and
         VEGF;
and incubating for 44-52 h, particularly for 46-50 h, more particularly for ˜48 h,
j) keeping the cells in the rotating incubator for a period of 5 to 7 days, particularly for a period of ˜6 days, wherein there is a continuous flow of fresh medium 6 or the medium is replaced regularly with fresh medium 6, particularly is replaced 2-4 times, more particularly 3 times, with each medium replacement after 44-52 h, particularly after 46-50 h, more particularly after ˜48 h;
k) harvesting the cardiac tissue organoid.

2. The method according to item 1, wherein medium 0 comprises
30-250 μg/L, particularly 50-200 μg/L, more particularly ˜100 μg/L FGF2,
10-30 mg/L, particularly 15-25 mg/L, more particularly ˜20 mg/L insulin, and
0.5-5 μg/L, particularly 1-3 μg/L, more particularly ˜2 μg/L TGFβ1.

3. The method according to any one of the preceding items, wherein medium 1 comprises
30-70 ng/ml, particularly 40-60 ng/ml, more particularly ˜50 ng/ml Activin A,
0.5-4 ng/ml, particularly 1-3 ng/ml, more particularly ˜2 ng/ml BMP-4,
3-7 ng/ml, particularly 4-6 ng/ml, more particularly ˜5 ng/ml FGF, 0.25-3 μmol/L, particularly 0.5-2 μmol/L, more particularly ˜1 μmol/L CHIR, and
25-200 μmol/L, particularly 50-150 μmol/L, more particularly ˜100 μmol/L ASC-2-P.

4. The method according to any one of the preceding items, wherein medium 2 comprises
3-7 ng/ml, particularly 4-6 ng/ml, more particularly ˜5 ng/ml Activin A,
4-20 ng/ml, particularly 5-15 ng/ml, more particularly ˜10 ng/ml BMP-4,
3-7 ng/ml, particularly 4-6 ng/ml, more particularly ˜5 ng/ml FGF,
0.25-3 μmol/L, particularly 0.5-2 μmol/L, more particularly ˜1 μmol/L CHIR, and
25-200 μmol/L, particularly 50-150 μmol/L, more particularly ˜100 μmol/L ASC-2-P.

5. The method according to any one of the preceding items, wherein medium 3 comprises
2-8 μmol/L, particularly 3.5-6.5 μmol/L, more particularly ˜5 μmol/L IWP4, and
50-400 μmol/L, particularly 100-300 μmol/L, more particularly ˜200 μmol/L ACS-2-P.

6. The method according to any one of the preceding items, wherein medium 4 comprises
50-400 μmol/L, particularly 100-300 μmol/L, more particularly ˜200 μmol/L ACS-2-P.

7. The method according to any one of the preceding items, wherein medium 5 comprises
50-400 μmol/L, particularly 100-300 μmol/L, more particularly ˜200 μmol/L ACS-2-P,
20-100 nmol/L, particularly 30-70 nmol/L, more particularly ˜50 nmol/L VEGF, and
5-100 nmol/L, particularly 10-50 nmol/L, more particularly ˜25 nmol/L FGF.

8. The method according to any one of the preceding items, wherein medium 6 comprises
30-350 μmol/L, particularly 80-250 mol/L, more particularly ˜160 μmol/L ACS-2-P,
1-15 ng/ml, particularly 3-10 ng/ml, more particularly ˜5 ng/ml EGF;
3-20 ng/ml, particularly 5-15 ng/ml, more particularly ˜10 ng/ml FGF;
10-30 ng/ml, particularly 15-25 ng/ml, more particularly ˜20 ng/ml IGF; and
0.1-1.5 ng/ml, particularly 0.3-1 ng/ml, more particularly ˜0.5 ng/ml VEGF.

9. The method according to any one of the preceding items, wherein each medium 1 to 6 comprises
RPMI 1640 supplemented with a vitamin and amino acid mix (Glutamax),
1 mMol/L sodium pyruvate,
100 U/mL penicillin,
100 μg/mL streptomycin, and
3% (volume/volume) of a cell growth supplement (B27 supplement).

10. The method according to any one of the preceding items, wherein in step a of providing the iPSCs, 100.000-600.000 cells/well or 500-2000 cells/microwell are provided.

11. The method according to any one of the preceding items, wherein said cardiac tissue organoid consists of human cells.

12. A cardiac tissue organoid obtained by the method of any one of the preceding items.

13. A cardiac tissue organoid obtainable by the method of any one of the preceding items 1 to 11.

14. The cardiac tissue organoid according to item 12 or 13 comprising cardiomyocytes;
endothelial cells;
fibroblasts;
smooth muscle cells;
pericytes;
neurons/sino-atrial node cells; and
macrophages.

The cardiac tissue organoid according to item 12 to 14, wherein the cardiac tissue organoid is in a mature phenotypic status characterized by
a rod shape;
a polarized structure; and
a sarcomeric structure of cardiomyocytes.

16. The cardiac tissue organoid according to item 12 to 15, wherein the cardiac tissue organoid is in a mature structural status characterized by a decreased ratio of mRNA expression levels of the mature cardiac organoid (day 35 after cardiomyocytes were made) relative to an immature cardiac organoid (d0—the first day after cardiomyocytes were made) for the following markers:
MYH6/MYH7, particularly at a ratio of 0.1;
MLC2a/MLC2v, particularly at a ratio of 0.001;
TNNI1/TNNI3, particularly at a ratio of 0.3;
and
TTN-N2B/TTN-N2BA, particularly at a ratio of 0.01.

17. The cardiac tissue organoid according to item 12 to 16, wherein the cardiac tissue organoid is in a mature functional status characterized by
an mRNA expression level of CAV3 (normalized to HPRT) of 0.01, particularly of 0.04.

18. The cardiac tissue organoid according to item 12 to 17, wherein the cardiac tissue organoid is in a mature metabolic status characterized by
an mRNA expression level of PKM2 (normalized to HPRT) of 2, particularly of 0.8.

19. The cardiac tissue organoid according to item 12 to 18 comprising
epicardium;
myocardium;
endocardium;
cardiac lumen; and
a three-dimensional vascular network.

20. The cardiac tissue organoid according to items 12 to 19 for use as a tissue implant or for use in regenerative medicine.

21. A method for screening a drug comprising the steps:
producing a cardiac tissue organoid according to the method of items 1 to 11,
contacting the cardiac tissue organoid with a drug of interest,
determining an effect of the drug on the cardiac tissue organoid.

22. The method according to item 21, wherein said effect of the drug on the cardiac tissue organoid is determined as one or several effects selected from:
Cell viability;
Cytotoxicity;
Cardiomyocyte cell death;
Proliferation;
Contractility;
Mitochondrial activity;
Metabolism.

Wherever alternatives for single separable features such as, for example, a growth factor or a concentration are laid out herein as “embodiments”, it is to be understood that such alternatives may be combined freely to form discrete embodiments of the invention disclosed herein. Thus, any of the alternative embodiments for a growth factor may be combined with any of the alternative embodiments of a concentration mentioned herein.

The invention is further illustrated by the following examples and figures, from which further embodiments and advantages can be drawn. These examples are meant to illustrate the invention but not to limit its scope.

### DESCRIPTION OF THE FIGURES

FIG. 1 shows the distribution of different cell types in cardiac organoid. Cardiac organoids were harvested and cryo-sectioned. After that the organoid sections were stained for DAPI, VE-Cadherin, and smooth muscle actin (SMA), showing distribution of nuclei, endothelial cells, and smooth muscle cells/pericytes, respectively. Magnified inlet overlay depicts co-localization and coverage of endothelia with smooth muscle cells/pericytes, indicative of mature blood vessel formation. Scale bar: 100 μm.

FIG. 2 shows shows the vascularization of cardiac organoid. Cardiac organoids were harvested and stained for DAPI (nuclei), α-Actinin (Cardiomyotes), SMA (smooth muscle cells/pericytes), and VE-cadherin (endothelial cells). Cardiomyocytes, smooth muscle cells/pericytes, and endothelial cells intermingle, yet form cell clusters at certain foci, as is observed in the native myocardium. VE-Cadherin stains endothelial cells, showing development of a vascularized network within organoids, further detailed by 3D reconstruction depicting blood vessel organization and lumen formation. Scale bar: 100 μm.

FIG. 3 shows the neurogenesis of cardiac organoid. Cardiac organoids were harvested and stained for stained for DAPI, pan-neuronal marker (Neuro-Pan) and Phalloidin, showing development of neurons on the organoid epicardium, as localized in native heart tissue. Scale bar: 100 μm.

FIG. 4 shows the formation of cardiac tube. Cardiac tissue organoids stained for DAPI, α-Actinin (CMs) and Phalloidin, showing development of epicardium, myocardium, endocardium and lumen. Scale bar: 100 μm.

FIG. 5 shows the maturity status of the organoids after 35 days of culture by immunofluorescent staining. Cardiac tissue organoids were stained for DAPI, α-Actinin (CMs) and Phalloidin. The 3D rendition of immunofluorescent image shows the rod-shape and alignment of cardiomyocytes, which indicates the mature cells. The magnified inlet shows the similarity between TrueCardium and human heart slide which was presented in an article. The reference image was taken from Watson, et al. (2017) Nature Protocols, 12(12), 2623-2639.

FIG. 6 shows the maturity status of the organoids by quantifying mRNA expression. During the cardiogenesis, the switching of isoforms of different genes related to myofibril assembly as MYH6/MYH7 (Myosin heavy chain), MLC2a/MLC2v (Myosin light chain), TNNI3/TNNI1 (Troponin I) and TTN N2B/TTN N2BA (Titin) was previously reported. Therefore, Cardiac organoids of day 0 and day 35 were harvested and total RNA was isolated for qPCR assay. mRNA expression level of above-mentioned pairs of genes was quantified. The upper panel shows the of switch in isoform expression in cardiac organoids from day 0 to day 35. The results were similar with previous published data which are shown in the lower panels. Previously published data relating to relative “gene expression” of MTH6 and MYH7 (FIG. 6., lower left panels) stems from Nunes SS, et al. Nat Methods. 2013; 10:781-787. Previously published data relating to “relative gene fraction” of MLC2a+2v and MLC2v (FIG. 6, lower right panel) stems from Shadrin IY, et al. Nat Commun. 2017; 8:1825. Previously published data relating to “Normalized to adult Human left ventricles”, “TNNI3/1 Ratio” and “TNNI Isoform content” (FIG. 6 (continued), lower left panels) stems from Shadrin IY, et al. Nat Commun. 2017; 8:1825; Mills RJ, et al. Proc Natl Acad Sci USA. 2017;114:E8372—E8381; Data from: Bedada FB, et al.Stem Cell Reports. 2014 Oct. 14; 3(4):594-605. Previously published data relating to “TTN N2B Rel CTRL” (FIG. 6 (continued), lower right panel) stems from Mills RJ, et al. Proc Natl Acad Sci USA. 2017;114:E8372—E8381.

FIG. 7 shows the maturity status of the organoids by quantifying mRNA expression. During the cardiac maturation, the expression of gene related to Calcium handling as CAV3 (Caveolin 3) was previously proved. Therefore, mRNA expression level of CAV3 in Cardiac organoids of day 0 and day 35 were quantified. The upper panel shows upregulation of CAV3 in cardiac organoid after 35 days of culture, which implies the maturation of the model. The result is in agreement with the in vivo results of CAV3 expression which were previously published. Previously published data relating to “Fold Change Normalized to DO” stems from Bosman et al. Stem Cells Dev. 2013 Jun. 1; 22(11):1717-27. Previously published data relating to “Fold change (log₂) compared to immature hiPSCs-CMs” stems from Ronaldson-Bouchard et al. Nature. 2018 Apr;556(7700):239-243.

FIG. 8 shows the maturity status of the organoids by quantifying mRNA expression. During the cardiac maturation, the metabolism of cardiomyocytes changes from glycolysis to fatty acid oxidation which was shown by the downregulation of PKM2 (Pyruvate kinase isozymes M2). Therefore, mRNA expression level of PKM2 in Cardiac organoids of day 0 and day 35 were quantified. The result in the upper panel shows downregulation of PKM2 in cardiac organoid after 35 days of culture, which implies the maturation of the model. The result is in line with the published data as the PKM2 expression at protein level during the heart development, which is shown in the lower panel (Gao Z and Cooper TA. Proc Natl Acad Sci USA. 2013 Aug. 13; 110(33):13570-5).

FIG. 9 shows the maturity status of the organoids by quantifying mRNA expression. As reported in the literature, the cardiac fetal/postnatal gene program (NPPA and NPPB) is repressed during the cardiac maturation. mRNA expression level of NPPA and NPPB in Cardiac organoids of day 0 and day 35 were quantified. The downregulation of both NPPA and NPPB (upper and middle panels) in cardiac organoid after 35 days of culture were observed, which is comparable to the literature (lower panels).

FIG. 10 shows different cell densities of EB affect contractility. EBs with different cells densities (from 300 cells to 2000 cells/EB) were generated and differentiated into cardiac organoids. From the initial density of 1000 cells/EB, organoids show very low or no contractility. The intitial density of 500 cells/EB was chosen for further differentiation.

FIG. 11 shows VEGF and FGF2 enhance vascularization. Cardiac organoids cultured with or without Medium 5 were stained for DAPI, VE-Cadherin and α-Actinin. In Medium 5, endothelial cells showed remarkable proliferation, which further formed vasculature network within organoid. Scale bar: 100 μm.

FIG. 12 shows early addition of VEGF and FGF2 affect cardiomyocytes' maturation. Cardiac organoids cultured with Medium 5 either at early and late time point were observed for contractility and stained for DAPI, VE-Cadherin and α-Actinin. Organoids with early addition of Medium 5 showed lower contractility and lower organization of sarcomeres, which indicated an affect on the maturation of cardiomyocytes. Scale bar: 20 μm.

FIG. 13 Cell types distribution by fraction of cells in TrueCardium organoids. Each data point represents immunofluorescent quantification for the respective cell type in a single organoid. The data shown is derived from a total of n≤15 independent experiments.

FIG. 14 Cell types distribution by area in TrueCardium organoids. Each data point represents immunofluorescent quantification for the respective cell type in a single organoid. The data shown is derived from a total of n≤15 independent experiments.

FIG. 15 Organoid—Tissue integration. GFP-expressed cardiac organoids co-cultured with fresh harvested adult rat ventricular tissue. After 20 days, co-cultured tissue stained for nuclei (DAPI) and endothelial cells (1134). Magnified inlet overlay shows the joining of vessels (arrow) from organoid (co-localization of GFP and IB4) and tissue (IB4), indicative of the integration of organoids into natural tissue. Scalebar: 100 μm.

FIG. 16 compares the method of the invention (GB) with methods disclosed in prior art documents (D2: EP 3540046 A1; D3: Richards et al. Biomaterials 142 (2017) 112-123; D4: WO 2016183143 A1; D6: EP 3882341 A1).

### EXAMPLES

Methods

TrueCardium generation

Human induced pluripotent stem cells (hiPSCs) were used for TrueCardium generation. In brief, 500 hiPSCs were cultured on ultra-low-attachment surface in medium TeSR™-E8™ at 37° C. and 5% CO₂ at humidified atmosphere to form embryoid body (EB). After 2 days, EBs were differentiated to cardiac organoids (COs) by replacing medium every 48 hours with medium 1, medium 2, medium 3 (2 times) and medium 4. COs were maintained in medium 4 for 10 days by refreshing medium every second day. Medium 4 was then replaced by medium 5 for 4 days with refreshing medium every 48 h. After that COs were transferred to rotating incubator with medium 6 for 6 days with refreshing medium every second day. COs were then ready to be harvested.

Cell density of iPS cells:
Optimize from 500 to 3000 iPSCs per EB
Density of 500 iPSCs per EB was chosen as it gave best result in organoid formation.

Timing:
Day −2: EB formation from iPSCs in microwell
Day 0: Add Medium 1 supplemented with Matrigel PSC grade (dilution 1:100)
After exact 48 h (day 2): Add Medium 2
After exact 48 h (day 4): Add Medium 3
After exact 48 h (day 6): Change fresh Medium 3
After exact 48 h (day 8): Add Medium 4
From day 8 to day 18: Change fresh Medium 4 every second day
Day 18: Add Medium 4 supplemented with 50 nM hVEGF and 25 nM hFGF
After exact 48 h (day 20): Change fresh Medium 4 supplemented with 50 nM hVEGF and 25 nM hFGF
After exact 48 h (day 22): transfer Cardiac organoids to rotating incubator and add Mix Medium (Medium 4: EGM2 (without FBS) at ratio 4:1)
From day 22 to day 28: maintain Cardiac organoids in rotating incubator. Change Mix Medium every second day
Day 29: Harvest cardiac organoid

Fluorescence Immunohistochemistry

COs were collected and fixed with 4% PFA over night at 4° C. Whole COs as well as cryo-sections of COs were used for fluorescence immunohistochemistry.

For whole mount staining, COs were permeabilized with 1% Triton X-100 in PBS for 45 minutes at room temperature followed by blocking with 5% horse serum in PBS for 45 minutes at room temperature. Primary antibodies were diluted 1:200 in 2% horse serum in PBS with 0,002% Triton X100 and incubated over night at 4° C. Before using the secondary antibody, COs were washed six times with PBS containing 0.002% Triton X-100 (referred as PBT from here after) for 4 hours at room temperature. Secondary antibodies (1:500) and DAPI (1:100) were diluted in 2% horse serum in PBS with 0,002% Triton X100 together and incubated for 4 hours at room temperature in the dark. After washing COs again three times with PBT, COs were mounted with ProLong Gold Antifade Mountant and analysed using the confocal microscope.

For cryo-section staining, cryo-sections were prepared by embedding fixed COs in O.C.T medium, snap-freezing them in liquid nitrogen and cutting them in 20 μm thick sections. Sections were permeabilized and blocked for one hour in 0.1% Triton X100 (in PBS+BSA). Primary antibody incubated with the sections diluted 1:50-1:100 in 1 mM MgCl₂, 1 mM CaCl₂), 0.1 mM MnCl₂, 1% Triton X-100 or 0.2% saponin over night at 4° C. The next day, sections were washed trice with 0.1% Triton X100/PBS or 0.2 saponin (five minutes each) and incubated with the secondary antibody (1:500) and DAPI (1:100) diluted in 0.1% Triton X100 or 0.2% saponin (in PBS) for one hour at room temperature in the dark. Finally, sections were washed again trice with 0.1% Triton X100/PBS or 0.2% saponin and mounted with mounting medium.

RNA Isolation and Quantification

COs were collected into Lysing Matrix D tubes (MP Biomedical) and 700 μL of TriFast (VWR; 3010-100ML) was added. Cells and tissues were homogenized 3 times for 20s following by 5 min. pause on ice. Total RNA was purified with the Direct-zolTM RNA MicroPrep kit (Zymo Research; R2060). The RNA concentration was determined by measuring absorption at 260 nm and 280 nm with the NanoDrop OND 2000-spectrophotometer (PeqLab).

cDNA synthesis and quantitative Polimerase chain reaction (qPCR) cDNA was synthesized from 500 ng of total RNA with EcoDry Premix RNA to cDNA (Random Hexamers) (Clontech; 639546). qPCR was performed using 10 μL of iTaq™ Universal SYBR ° Green Supermix (Bio-Rad; 172-5124), 1 μL of 10 μmol/L forward and reverse primer each, 1 μL of cDNA template and 7 μL of H₂O in a Bio-Rad CFX96 Connect Real-Time PCR system. Primers using to quantify mRNA expression were ordered from Metabion.

Integration of Cardiac Organoid into Mice Hearts

Cardiac organoids were incubated with concentrated GFP Lentivirus overnight at 37° C., 5% CO₂. The virus supernatant was then replaced with medium 6 and 50% of medium was changed every second day for 3 days.

The adult rat heart was isolated and soaked in ice-cold sterile 0,02M BDM (2,3-Butanedione monoxime) in HBSS. The ventricular tissue was then sectioned into small blocks and maintained in medium 6 in ultra-low-attachment 96-well-plate overnight at 37° C., 5% CO₂. The next day GFP-labelled organoids were transferred into the well with tissue for co-culture. Medium 6 were changed every day for 20 days. The fused tissue-organoids were then collected, fixed with PFA 4% and cryo-sectioned for 50 μm. The sections of tissue-organoid were used later for immunofluorescence staining.

Material

| | | |
|---|---|---|
| TeSR ™-E8 ™ Kit | Stemcell Technologies | Cat 05940 |
| * RPMI 1640 with Glutamax | Invitrogen | Cat 61870-010 |
| * Endothelial Cell Growth | Promocell | Cat C-22111 |
| Medium 2 kit | | |
| 100 mM sodium pyruvate | Invitrogen | Cat 11360 |
| 100X penicillin/streptomycin | Invitrogen | Cat 15140 |
| BMP4 | R&D systems | Cat 314-BP |
| Activin A | R&D systems | Cat 338-AC |
| FGF-2 | Miltenyi Biotech | Cat 130-093-841 |
| VEGF | R&D systems | Cat 293-VE |
| IWP4 | Stemgent | Cat 04-0036 |
| CHIR99021 | Stemgent | Cat 04-0004 |
| Rock Inhibitor Y27632 | Stemcell Technologies | Cat 72302 |
| B27 | Gibco | Cat 17504044 |
| DMSO | Sigma | Cat D2650 |

L-ascorbic acid 2 phosphate sesquimagnesium salt hydrate (ASC),

| | | |
|---|---|---|
| | Sigma | Cat A8960-5G |
| Matrigel | Corning | Cat 354277 |

* Endothelial Cell Growth Medium 2 contains (in 500 mL of medium - information available https://www.promocell.com/f/product-information/manual/C-22111.pdf):
Epidermal Growth Factor (recombinant human) 5 ng/ml
Basic Fibroblast Growth Factor (recombinant human) 10 ng/ml
Insulin-like Growth Factor (R3 IGF-1) 20 ng/ml
Vascular Endothelial Growth Factor 165 (recombinant human) 0.5 ng/ml
Ascorbic Acid 1 μg/ml
Heparin 22.5 μg/ml
Hydrocortisone 0.2 μg/m
* RPMI 1640 with Glutamax contains (in 500 mL of medium - information available

https://wwwthermofishercom/de/de/home/technical-resources/media-formulation 122. html)

Media info:
Basal medium:
   RPMI 1640 with Glutamax
   1% of 100X sodium pyruvate
   1% of 100X penicillin/streptomycin
   3% B27 supplement
Medium 1:
   Basal medium
   50 ng/mL of Activin A
   2 ng/mL of BMP-4
   5 ng/mL of hFGF
   1 μM CHIR
   100 μM ACS-2-P
Medium 2:
   Basal medium
   5 ng/mL of Activin A
   10 ng/mL of BMP-4
   5 ng/mL of bFGF
   1 μM CHIR
   100 μM ACS-2-P
Medium 3:
   Basal medium
   5 μM IWP4
   200 μM ACS-2-P
Medium 4:
   Basal medium
   200 μM ACS-2-P

Results

### Example 1: Establishment of Cardiac Organoids

To establish the cardiac organoid, Human induced pluripotent stem cells (hiPSCs) were used for organoid generation. In brief, 500 hiPSCs were cultured on ultra-low-attachment surface in medium TeSR™-E8™ at 37° C. and 5% CO₂ at humidified atmosphere to form embryoid body (EB). After 2 days, EBs were differentiated to cardiac organoids (COs) by replacing medium every 48 hours with medium 1, medium 2, medium 3 (2 times) and medium 4. COs were maintained in medium 4 for 10 days by refreshing medium every second day. Medium 4 was then replaced by medium 5 for 4 days with refreshing medium every 48 h. After that COs were transferred to rotating incubator with medium 6 for 6 days with refreshing medium every second day. COs were then ready for further assay.

### Example 2: Self-Differentiation and Distribution of Important Cell Types

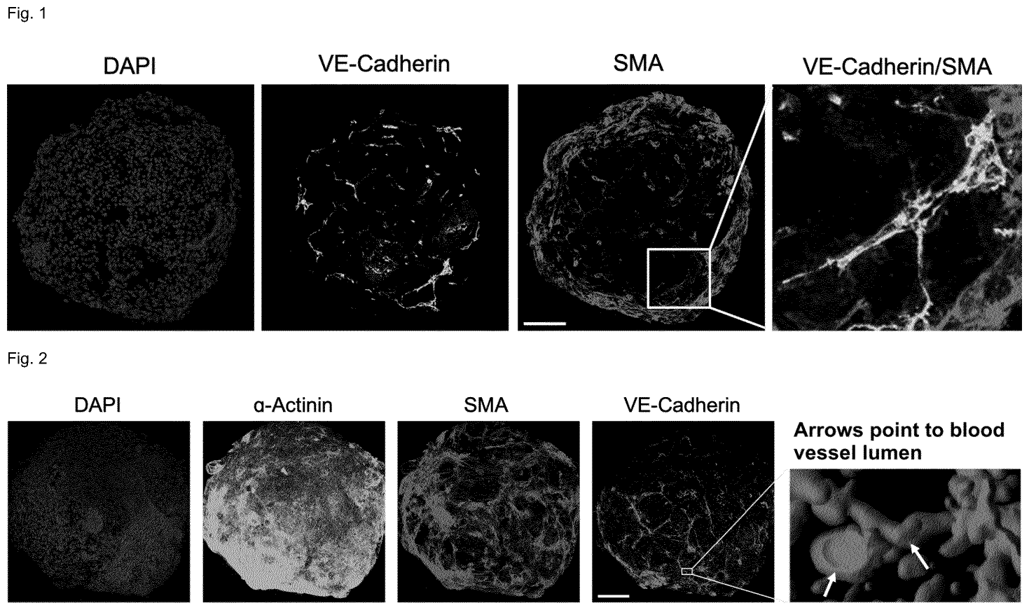

It was assessed if different cell types were self-differentiated and—distributed in the cardiac organoids as similar to native tissue. At first to observe if the cardiac organoids had endothelial cells and other supporting cells for building a vascular network, cryo-sections were stained with VE-Cadherin, a marker for endothelial cells (EC), and smooth-muscle-actin-α (α-SMA), a marker for smooth muscle cells and pericytes. Indeed, mature blood vessels were found in the cardiac organoid evidenced by an extensive endothelial signal and the co-localization of α-SMA and VE-Cadherin, which indicated the endothelia coverage by smooth muscle cells/pericytes (FIG. 1). Furthermore, a vascular network was developed within the organoid as shown by wholemount staining with VE-Cadherin and the 3D reconstruction depicted the lumen formation of the blood vessel (FIG. 2). In parallel, cardiomyocytes (CM) stained with α-Actinin, smooth muscle cells/pericytes and endothelial cells intermingled and formed cell clusters at certain foci which is observed in the native myocardium. Taken together, the data proves that the cardiac organoids self-generated a stable blood vessel network during the development which is similar to the native tissue.

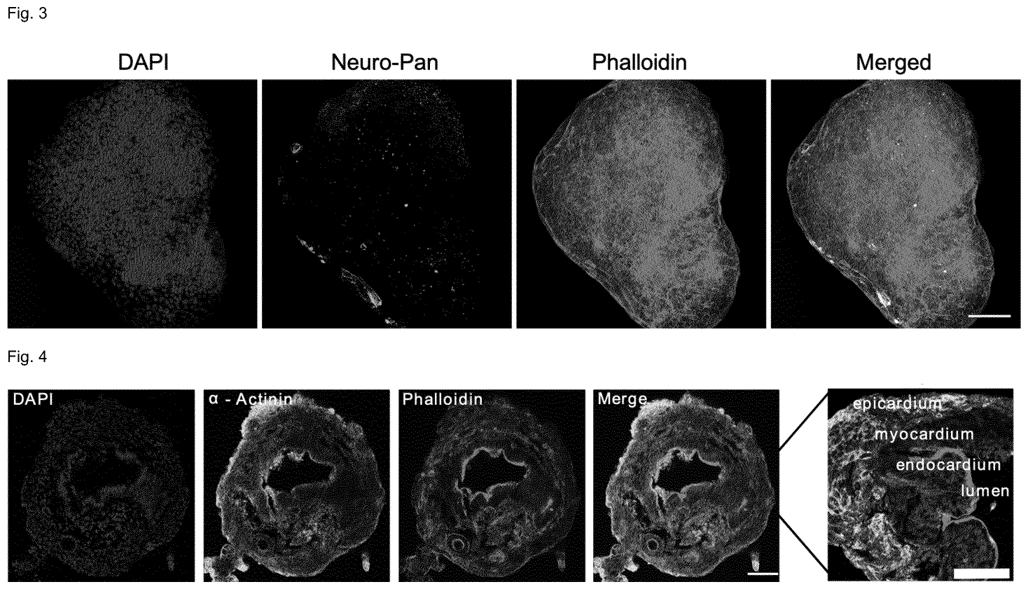

Beside the blood vessels, the native cardiac tissue is known to have a nervous system (Duraes Campos, Isabel et al. Journal of molecular and cellular cardiology vol. 119 (2018): 1-9.). Consistent with literature, the development of neurons was found on the organoid epicardium by IF staining with pan-neuronal marker (FIG. 3).

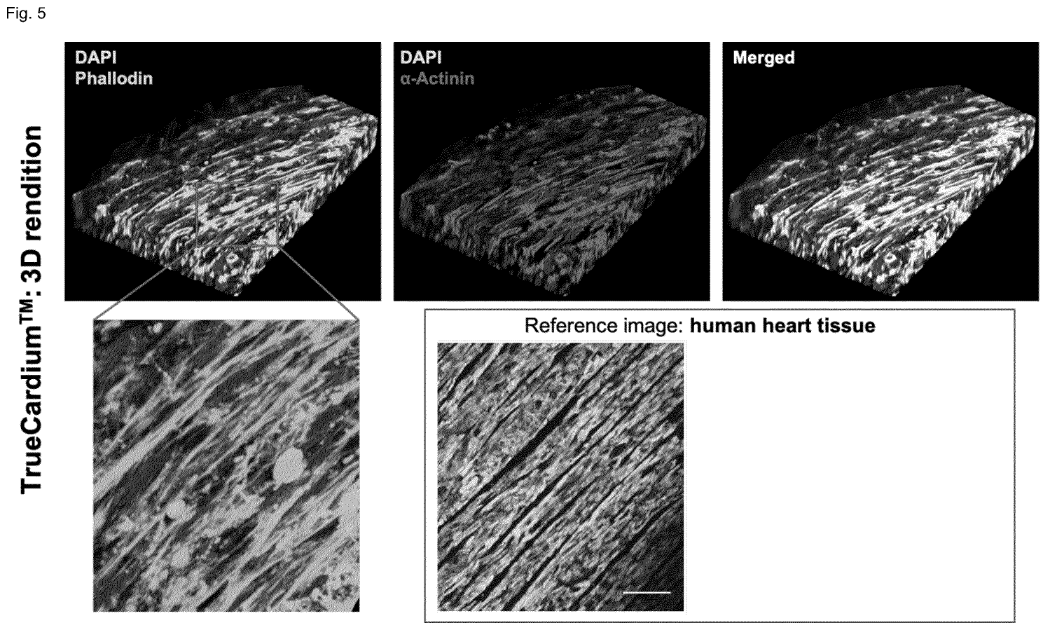

To discover if the cardiac organoids followed the heart development, the cryo-sections were stained for α-Actinin and Phalloidin (FIG. 4). The immunofluorescent signal revealed that the cardiac organoids were developing epicardium, myocardium, endocardium and lumen. The result was similar to the heart tube formation during the early heart development.

Taken together, the data demonstrate that cardiac organoids generated with the inventors' method have important cell types and distribution which is comparable to cardiac native tissue.

### Example 3: Maturity Status of Cardiac Organoids

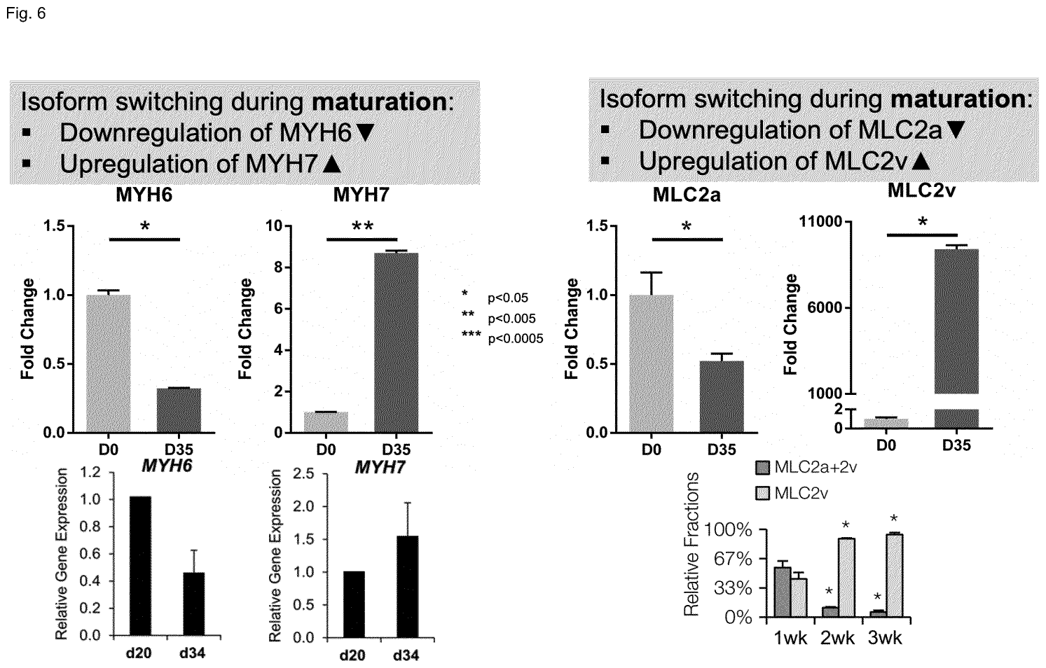

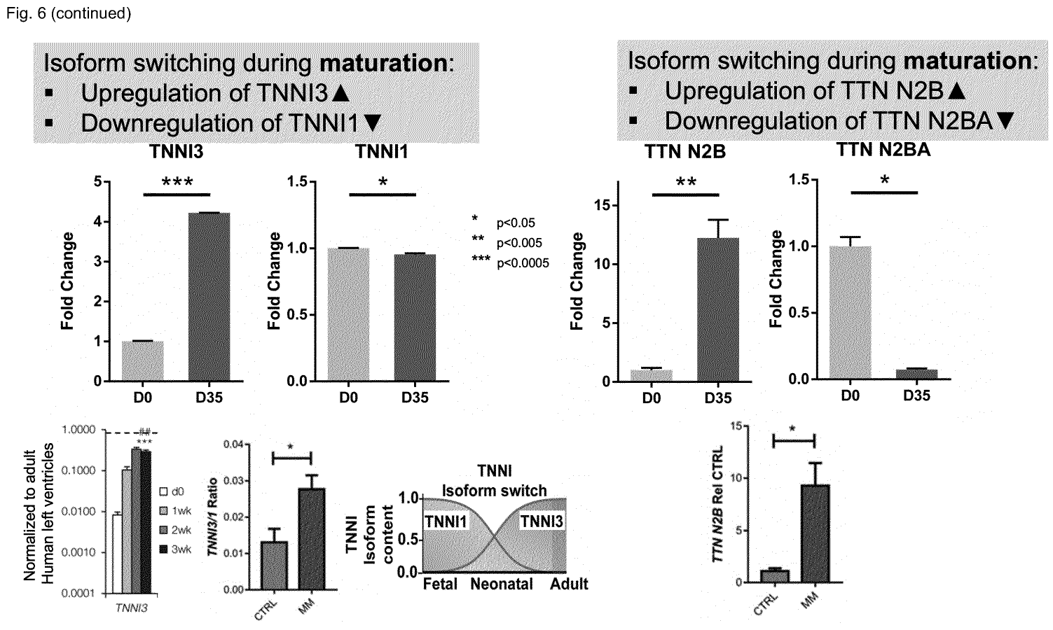

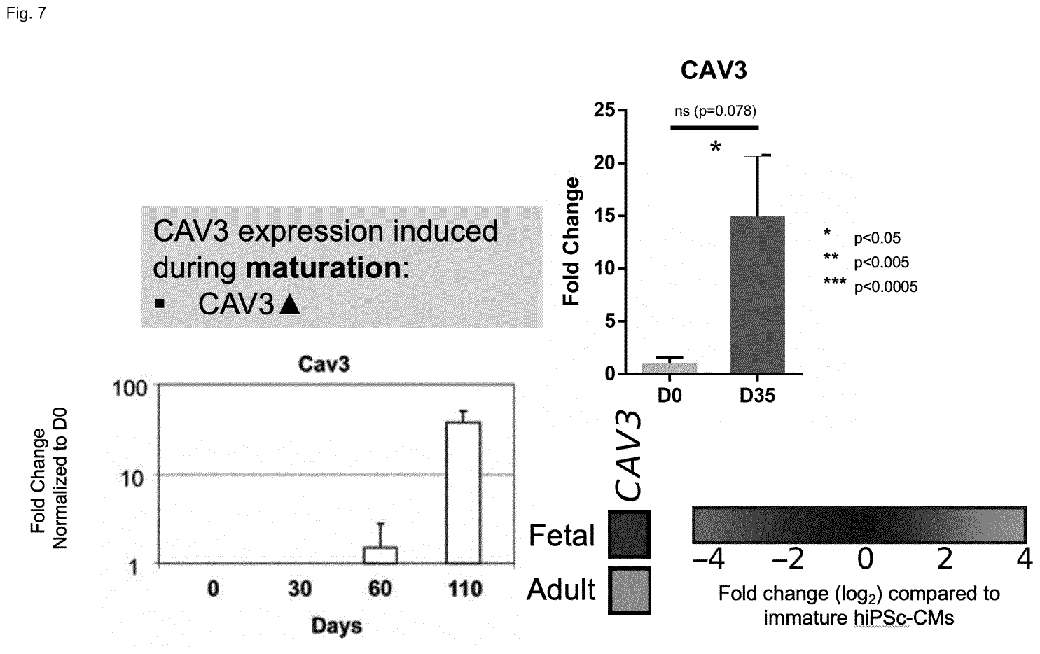

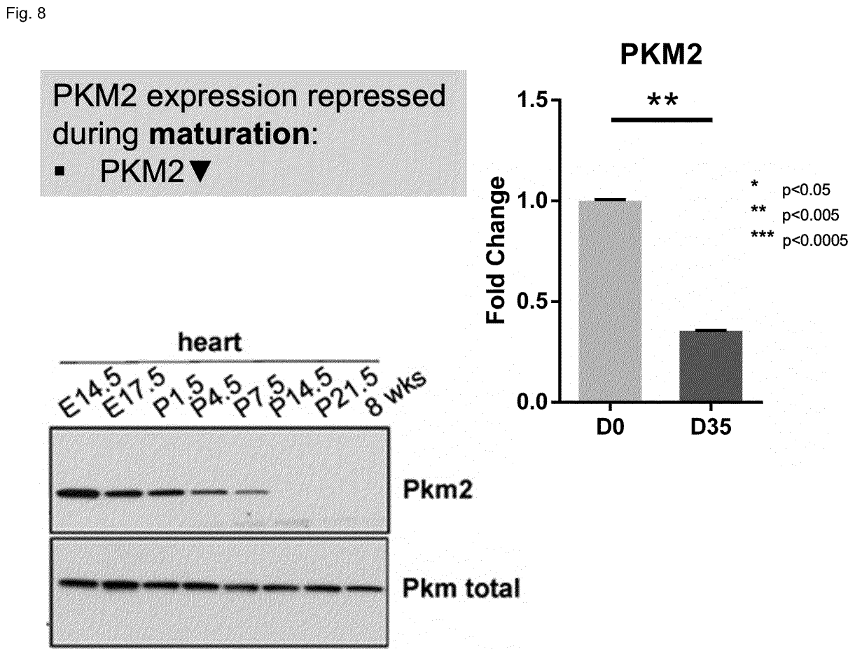

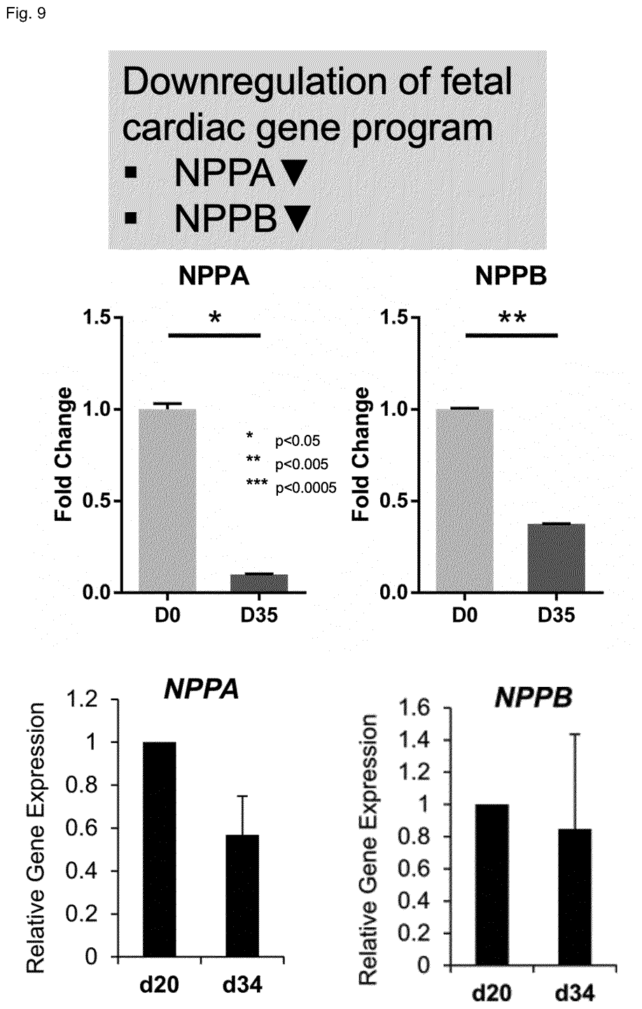

To evaluate the maturity status of cardiac organoids, at first immunofluorescent staining was performed for cardiomyocytes (FIG. 5). Consistent with the literature of in vivo heart tissue (Watson, S., Scigliano, M., Bardi, L et al. Nat Protoc 12,2623-2639 (2017).), cardiomyocytes from organoids after 35 days of culture showed the mature phenotypes evidenced by the rod-shape and alignment. Furthermore, the maturity status was assessed by quantifying mRNA expression for a range of maturation markers (FIG. 6-9, Table 1-2). Previous research proved that the switching of isoforms of genes related to myofibril assembly happened during the cardiogenesis (Nunes SS, et al. Nat Methods. 2013; 10:781-787; Shadrin I Y, et al. Nat Commun. 2017; 8:1825; Mills RJ, et al. Proc Natl Acad Sci USA. 2017;114:E8372—E8381). Consistently, the mRNA expression from organoids at day 0 and day 35 of culture showed the switching of isoforms as MYH6/MYH7 (Myosin heavy chain), MLC2a/MLC2v (Myosin light chain), TNNI3/TNNI1 (Troponin I) and TTN N2B/TTN N2BA (titin) (FIG. 6) towards an adult-type pattern. The data suggest that the cardiomyocytes in the aged organoids own the developed contractile structure (sarcomeres) which matches the observed phenotypic maturation (FIG. 5). Beside the structural maturation, cardiac organoids at day 35 also showed the functional maturation demonstrated by the upregulation of CAV3 (Ca²⁺ handling) which is in agreement with published in vivo results (Bosman et al. Stem Cells Dev. 2013 Jun. 1; 22(11):1717-27; Ronaldson-Bouchard et al. Nature. 2018 April; 556(7700):239-243) (FIG. 6). In term of metabolism, the mRNA expression level of PKM2 (Pyruvate kinase) was quantified. In line with the literature (Gao Z and Cooper TA. Proc Natl Acad Sci USA. 2013 Aug. 13; 110(33):13570-5; Rees ML, et al. Biochem Biophys Res Commun. 2015 Apr. 10; 459(3):430-6), the downregulation of PKM2 in cardiac organoids after 35 days of culture suggested that the metabolism of cardiomyocytes changes from glycolysis to fatty acid oxidation, which implies the metabolic maturation of the model. On the other hand, the cardiac fetal/postnatal genes (NPPA and NPPB) were reported being repressed during the cardiac maturation (Nunes SS, et al. Nat Methods. 2013; 10:781-787). Comparable to the literature, the downregulation of both NPPA and NPPB was observed indicating that cardiac organoids were no longer in the fetal, but in the mature state. Inclusively, the data proved that the inventors' cardiac organoid model is phenotypically, structurally, functionally and metabolically mature.

TABLE 1
CAV3 expression normalized to HPRT

| | | |
|---|---|---|
| CAV3 | d0 | d35 |

| | |
|---|---|
| 0.003717 | 0.058873 |

TABLE 2
PKM-2 expression normalized to HPRT

| | | |
|---|---|---|
| PKM-2 | d0 | d35 |

| | |
|---|---|
| 4.130257 | 1.572945 |

### Example 4: VEGF and FGF2 Enhance Vascularization

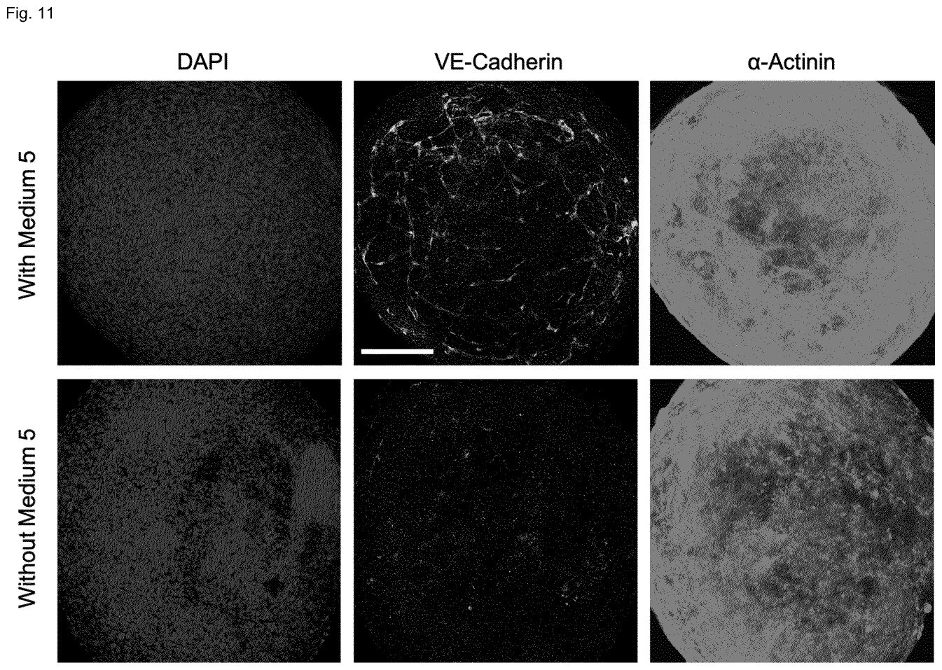

Cardiac organoids cultured with or without Medium 5 for 4 days. After 4 days, organoids were collected and stained for nuclei (DAPI), endothelial cells (VE-Cadherin) and cardiomyocytes (α-Actinin). The results showed that after 4 days in Medium 5 (containing VEGF and FGF2), endothelial showed strong proliferation and formed a massive vasculature network (FIG. 11).

### Example 5: Early addition of VEGF and FGF2 affect cardiomyocytes' maturation

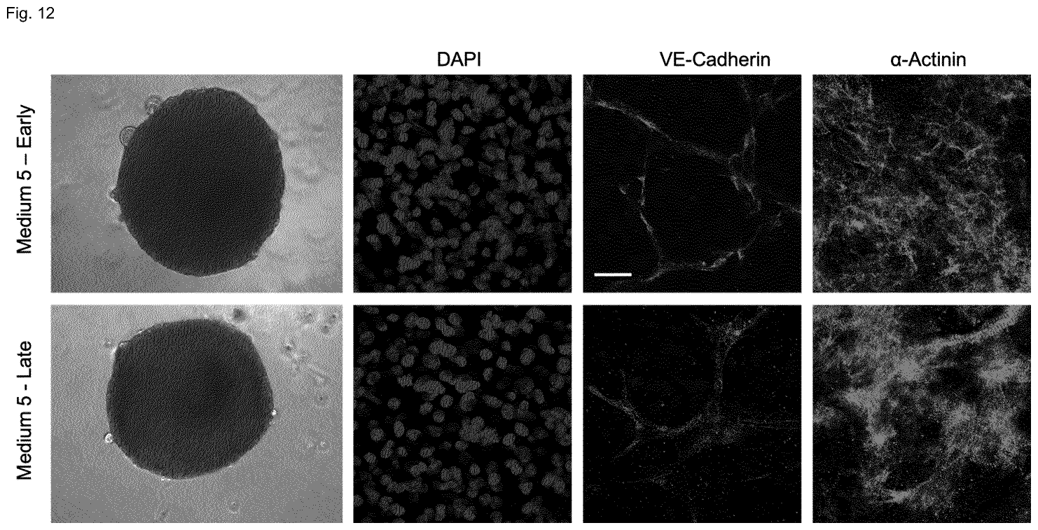

Cardiac organoids cultured with Medium 5 for 4 days starting from day 10 (early time-point) or day 20 (late time-point—chosen time-point for the method). After 4 days in Medium 5 from day the organoids were further maintained in medium 6. Meanwhile, other organoids were cultured in Medium 5 for 4 days following the method (start at day 20). At day 24, cardiac organoids from both cases were collected and observed for contractility under bright-field microscope. Cardiac organoids with early addition of Medium 5 (containing VEGF and FGF2) showed lower contractility (FIG. 12).

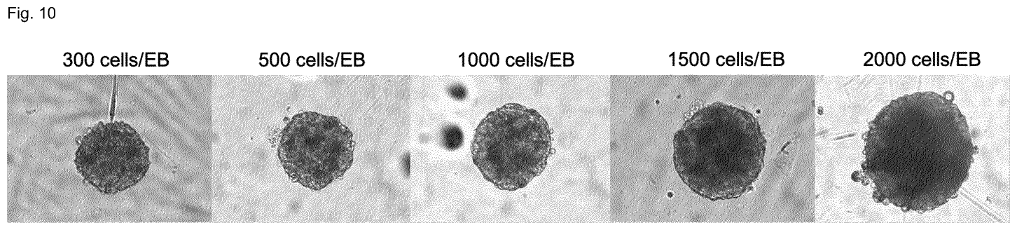

Organoids were then stained for nuclei (DAPI), endothelial cells (VE-Cadherin) and cardiomyocytes (α-Actinin). The cardiomyocytes in organoids with early addition of Medium 5 showed low organization of sarcomeres while in the later case the organization of sarcomeres were clearly enhanced, which is in agreement with the contractility (FIG. 12). Different cell densities of EB affect contractility (FIG. 10).

The result implies that early addition of medium with VEGF and FGF2 might affect the maturation of cardiomyocytes.

### Example 6: Characterization of cardiac organoids

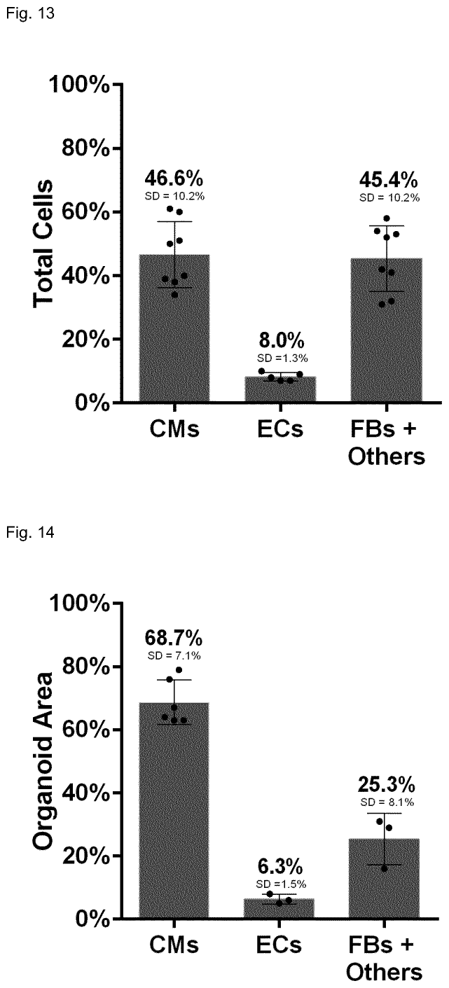

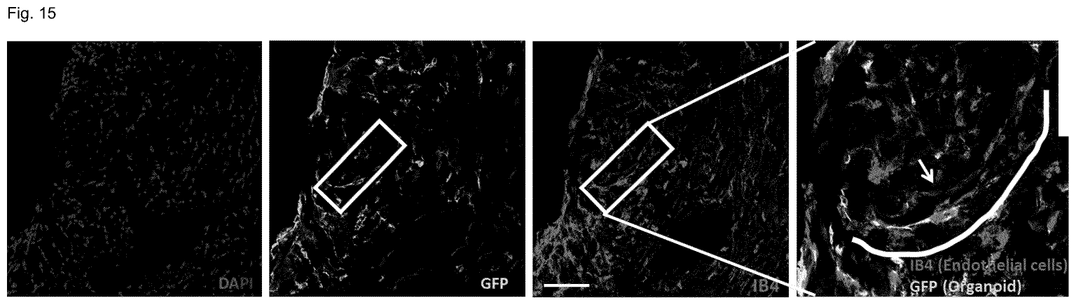

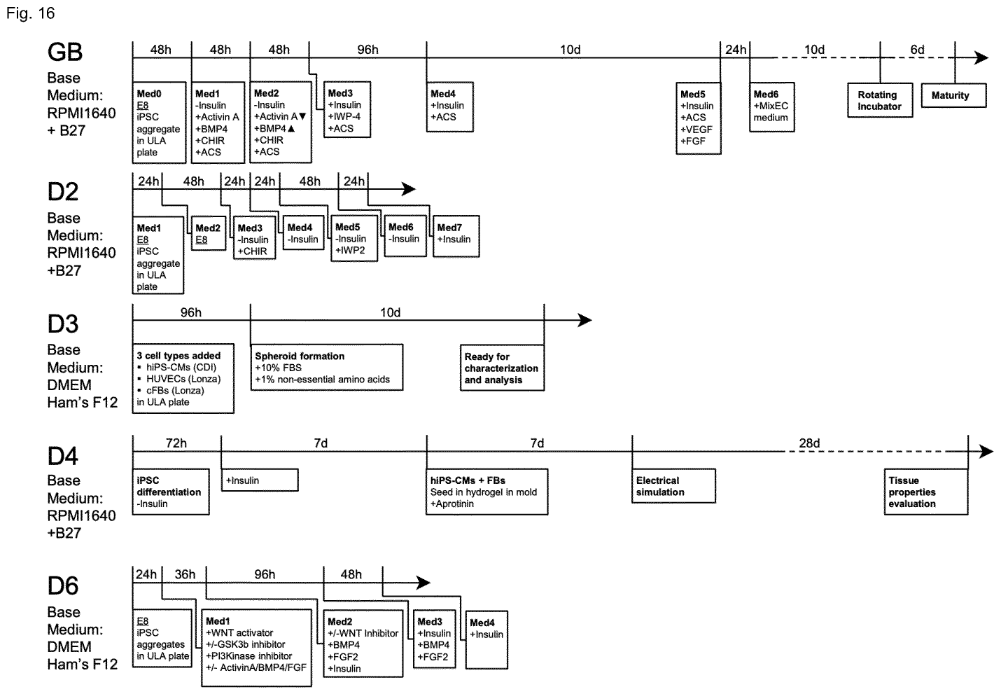

FIG. 13 depicts the cell type distribution of the respective cell types as a percentage of total cells. The three major cell types of the heart are Cardiomyocytes (CMs), Endothelial Cells (ECs), and Fibroblasts (FBs) and other cells (including pericytes, smooth muscle cells and other minority cell populations). Each data point represents immunofluorescent quantification for the respective cell type of a single organoid. The data shown is in FIG. 13 is derived from n≤5 independent experiments. The standard deviation (SD) is an indicator of inter-organoid variability. It is within a narrow range (up to 10.2%), indicating high reproducibility and consistency of cell types across organoids.

The spatial organization of cell types is highly consistent across organoids. In human heart sections the area fractions occupied by CMs and ECs are 70-80% and 3.2-5.3%, respectively. FIG. 14 shows the area coverage percentages in TrueCardium organoids which is directly comparable to and match the literature-reported proportions.

1. A method for production of a cardiac tissue organoid comprising the steps:
a) providing pluripotent stem cells, particularly induced pluripotent stem cells (iPSCs), in a well or a microwell, wherein the well or microwell comprises medium 0, and incubating said cells for 44-52 h, particularly for 46-50 h, more particularly for ˜48 h, wherein
medium 0 comprises:
FGF2,
insulin, and
TGF(31
and medium 0 does not comprise:
Activin A,
BMP-4,
CHIR,
ACS-2-P,
IWP4, and
VEGF;
b) subsequently, replacing the medium with medium 1, wherein
medium 1 comprises
Activin A,
BMP-4,
FGF, particularly hFGF,
CHIR, and
ACS-2-P;
and medium 1 does not comprise:
IWP4,
VEGF, and
TGF131
medium 1 is supplemented with a solubilized membrane preparation extracted from mammalian cells (Matrigel),
and incubating for 44-52 h, particularly for 46-50 h, more particularly for ˜48 h,
c) subsequently, replacing the medium with medium 2, wherein
medium 2 comprises
Activin A at a concentration which is lower than in medium 1, particularly Activin A at a concentration which is 5-20% (mass/volume), particularly 8-15% (m/v), more particularly ˜10% (m/v) of the concentration of Activin A in medium 1;
BMP-4 at a concentration which is higher than in medium 1, particularly BMP-4 at a concentration which is 500-2000% (m/v), particularly 800-1300% (m/v), more particularly ˜1000% (m/v) of the concentration of BMP-4 in medium 1;
FGF, particularly hFGF,
CHIR, and
ACS-2-P;
and medium 2 does not comprise:
IWP4,
VEGF,
insulin, and
TGF(31 and incubating for 44-52 h, particularly for 46-50 h, more particularly for ˜48 h,
d) subsequently, replacing the medium with medium 3, wherein
medium 3 comprises
insulin,
IWP4, and
ACS-2-P;
and medium 3 does not comprise:
VEGF,
Activin A,
BMP-4,
FGF,
CHIR, and
TGF(31
and incubating for 44-52 h, particularly for 46-50 h, more particularly for ˜48 h,
e) subsequently, repeating step d of incubating with medium 3 one time,
f) subsequently, replacing the medium with medium 4, wherein
medium 4 comprises
insulin, and
ACS-2-P;
and medium 4 does not comprise:
IWP4,
VEGF,
Activin A,
BMP-4,
FGF,
CHIR, and
TGF(31
and incubating for 44-52 h, particularly for 46-50 h, more particularly for ˜48 h,
g) repeating step f of incubating with medium 4 for 4-6 times, particularly 5 times;
h) subsequently, replacing the medium with medium 5, wherein
medium 5 comprises
insulin,
ACS-2-P,
VEGF, particularly hVEGF, and
FGF, particularly hFGF;
and medium 5 does not comprise:
IWP4,
Activin A,
BMP-4,
CHIR, and
TGF(31
and incubating for 44-52 h, particularly for 46-50 h, more particularly for ˜48 h,
i) subsequently, transferring cells to a rotating incubator and replacing the medium with medium 6, wherein
medium 6 comprises
insulin,
ACS-2-P, and
Endothelial Cell Growth Medium 2 (without FBS) comprising
EGF;
FGF;
IGF; and
VEGF;
and medium 6 does not comprise:
IWP4,
Activin A,
BMP-4,
CHIR, and
TGF(31
and incubating for 44-52 h, particularly for 46-50 h, more particularly for ˜48 h,
j) keeping the cells in the rotating incubator for a period of 5 to 7 days, particularly for a period of ˜6 days, wherein there is a continuous flow of fresh medium 6 or the medium is replaced regularly with fresh medium 6, particularly is replaced 2-4 times, more particularly 3 times, with each medium replacement after 44-52 h, particularly after 46-50 h, more particularly after ˜48 h;
k) harvesting the cardiac tissue organoid.

2. The method according to claim 1, wherein medium 0 comprises
30-250 μg/L, particularly 50-200 μg/L, more particularly ˜100 μg/L FGF2,
10-30 mg/L, particularly 15-25 mg/L, more particularly ˜20 mg/L insulin, and
0.5-5 μg/L, particularly 1-3 μg/L, more particularly ˜2 mg/L TGF(31.

3. The method according to claim 1, wherein medium 1 comprises
30-70 ng/ml, particularly 40-60 ng/ml, more particularly ˜50 ng/ml Activin A,
0.5-4 ng/ml, particularly 1-3 ng/ml, more particularly ˜2 ng/ml BMP-4,
3-7 ng/ml, particularly 4-6 ng/ml, more particularly ˜5 ng/ml FGF,
0.25-3 μmol/L, particularly 0.5-2 μmol/L, more particularly ˜1 μmol/L CHIR, and
25-200 μmol/L, particularly 50-150 μmol/L, more particularly ˜100 μmol/L ASC-2-P.

4. The method according to claim 1, wherein medium 2 comprises
3-7 ng/ml, particularly 4-6 ng/ml, more particularly ˜5 ng/ml Activin A,
4-20 ng/ml, particularly 5-15 ng/ml, more particularly ˜10 ng/ml BMP-4,
3-7 ng/ml, particularly 4-6 ng/ml, more particularly ˜5 ng/ml FGF,
0.25-3 μmol/L, particularly 0.5-2 μmol/L, more particularly ˜1 μmol/L CHIR, and
25-200 μmol/L, particularly 50-150 μmol/L, more particularly ˜100 μmol/L ASC-2-P.

5. The method according to claim 1, wherein medium 3 comprises
2-8 μmol/L, particularly 3.5-6.5 μmol/L, more particularly ˜5 μmol/L IWP4, and
50-400 μmol/L, particularly 100-300 μmol/L, more particularly ˜200 μmol/L ACS-2-P.

6. The method according to claim 1, wherein medium 4 comprises
50-400 μmon, particularly 100-300 μmon, more particularly ˜200 μmol/L ACS-2-P.

7. The method according to claim 1, wherein medium 5 comprises
50-400 μmon, particularly 100-300 μmon, more particularly ˜200 μmol/L ACS-2-P,
20-100 nmol/L, particularly 30-70 nmol/L, more particularly ˜50 nmol/L VEGF, and
5-100 nmol/L, particularly 10-50 nmol/L, more particularly ˜25 nmol/L FGF.

8. The method according to claim 1, wherein medium 6 comprises
30-350 μmon, particularly 80-250 mol/L, more particularly ˜160 μmon ACS-2-P,
1-15 ng/ml, particularly 3-10 ng/ml, more particularly ˜5 ng/ml EGF;
3-20 ng/ml, particularly 5-15 ng/ml, more particularly ˜10 ng/ml FGF;
10-30 ng/ml, particularly 15-25 ng/ml, more particularly ˜20 ng/ml IGF; and
0.1-1.5 ng/ml, particularly 0.3-1 ng/ml, more particularly ˜0.5 ng/ml VEGF.

9. (canceled)

10. The method according to claim 1, wherein in step a of providing the iPSCs, 100.000-600.000 cells/well or 500-2000 cells/microwell are provided.

11. The method according to claim 1, wherein said cardiac tissue organoid consists of human cells.

12. A cardiac tissue organoid obtained by the method of claim 1.

13. (canceled)

14. The cardiac tissue organoid according to claim 12 comprising
cardiomyocytes;
endothelial cells;
fibroblasts;
smooth muscle cells;
pericytes;
neurons/sino-atrial node cells; and
macrophages.

15. The cardiac tissue organoid according to claim 12, wherein the cardiac tissue organoid is in a mature phenotypic status characterized by
a rod shape;
a polarized structure; and
a sarcomeric structure of cardiomyocytes.

16. The cardiac tissue organoid according to claim 12, wherein the cardiac tissue organoid is in a mature structural status characterized by a decreased ratio of mRNA expression levels of the mature cardiac organoid (day 35 after cardiomyocytes were made) relative to an immature cardiac organoid (d0—the first day after cardiomyocytes were made) for the following
markers:
MYH6/MYH7, particularly at a ratio of ≤0.1;
MLC2a/MLC2v, particularly at a ratio of ≤0.001;
TNNI1/TNNI3, particularly at a ratio of ≤0.3;
and
TTN-N2B/TTN-N2BA, particularly at a ratio of ≤0.01.

17. The cardiac tissue organoid according to claims 12 to 16, wherein the cardiac tissue organoid is in a mature functional status characterized by
an mRNA expression level of CAV3 (normalized to HPRT) of ≥0.01, particularly of ≥0.04.

18. The cardiac tissue organoid according to claim 12, wherein the cardiac tissue organoid is in a mature metabolic status characterized by
an mRNA expression level of PKM2 (normalized to HPRT) of ≤2, particularly of ≤0.8.

19. The cardiac tissue organoid according to claim 12 comprising
epicardium;
myocardium;
endocardium;
cardiac lumen; and
a three-dimensional vascular network.

20. The cardiac tissue organoid according to claim 12 for use as a tissue implant or for use in regenerative medicine.

21. A method for screening a drug comprising the steps:
producing a cardiac tissue organoid according to the method of claim 1,
contacting the cardiac tissue organoid with a drug of interest,
determining an effect of the drug on the cardiac tissue organoid.

22. The method according to claim 21, wherein said effect of the drug on the cardiac tissue organoid is determined as one or several effects selected from:
Cell viability;
Cytotoxicity;
Cardiomyocyte cell death;
Proliferation;
Contractility;
Mitochondrial activity;
Metabolism.
